(19) United States
(12) Patent Application Publication
(10) Pub. No.: US 2008/0138873 A1
(43) Pub. Date: Jun. 12, 2008

(54) Yeast Strains With Improved Fructose Fermentation Capacity

(76) Inventors: Patrice Jacques Marie Pellerin, Laverune (FR); Bruno Blondin, Montpellier (FR); Jean-Marie Sablayrolles, Montpellier (FR); Carole Guillaume, Saint George d'Orques (FR)

Correspondence Address: NIXON & VANDERHYE, PC, 901 NORTH GLEBE ROAD, 11TH FLOOR, ARLINGTON, VA, 22203, US

(21) Appl. No.: 10/582,987

(22) PCT Filed: Dec. 20, 2004
(86) PCT No.: PCT/EP04/14577

§ 371 Date: Jul. 14, 2006

(30) Foreign Application Priority Data
Dec. 19, 2003 (EP) 03078992.9

(51) Int. Cl. C12P 1/02; C07K 14/00; C07H 21/00; C12N 1/00; C12N 15/01

(52) U.S. Cl. 435/171; 530/350; 536/23.74; 435/254.2; 435/440; 435/254.21

(57) ABSTRACT

The present invention relates to yeast strains with improved carbohydrate fermentation capacity, in particular to strains transformed with a gene encoding an improved hexose transporter gene, more in particular a mutated HXT3 gene.

### FIELD OF THE INVENTION

The present invention relates to yeast strains with improved carbohydrate fermentation capacity, in particular to strains transformed with a gene encoding an improved hexose transporter gene, more in particular a mutated HXT3 gene.

During the alcoholic fermentation of wine, hexoses such as glucose and fructose are converted into alcohol by microbial activity, in particular by yeast strains of the genus Saccharomyces. S. cerevisiae is a preferred yeast in wine-making and selected strains of S. cerevisiae are used as starters to inoculate grape musts and perform the alcoholic fermentation. Grape musts contain equivalent amounts of glucose and fructose whereas the level of total hexoses typically ranges from 160 to 300 g/L. S. cerevisiae is a glucophilic yeast, preferring glucose to any other carbon source that may be present in the growth substrate. As a result, the fructose/glucose ratio of the must progressively increases during the alcoholic fermentation. It has indeed been confirmed that after fermentation and in bottled wine, fructose is always found in larger amounts than glucose. During fermentation, a strong imbalance in the ratio of fructose and glucose is assumed to be a leading cause of stuck fermentation.

On a molecular level, the fermentation capacity of yeasts has been studied quite extensively. One of the early steps in the metabolism of sugars by the action of yeast is the transport of the sugars across the plasma membrane. Specific genes encoding transporters for different sugars are expressed in yeast. In Saccharomyces, the uptake of hexoses such as glucose and fructose, is mediated by specific hexose transporters that belong to a superfamily of monosaccharide facilitators (Reifenberger, E., Freidel, K. and Ciriacy, M. (1995) 16(1), 157-167). To date, more than sixteen genes encoding such genes, notably the so-called HXT-genes (which stands for hexose transport), have been identified. The expression of individual HXT-genes and homologues is dependent on environmental factors, such as the hexose concentration sensed by the yeast cell. It has been proposed that the uptake of hexoses is catalysed by two kinetically different systems (Bisson, L. F., Coons, D. M., Kruckeberg, A. L. and Lewis D. A. (1993) Crit Rev Biochem Mol Biol 28, 295-308; Lagunas, R. (1993) FEMS Microbiol Rev 104, 229-242)

One system has a high affinity for hexoses. This high affinity component is absent in cells growing in relatively high hexose concentrations, e.g. 2% glucose. Under these conditions the yeast cell expresses low affinity transporters. Construction of mutant yeast strains lacking multiple HXT genes made it possible to identify the main glucose transporters in yeast (Reifenberger, E., Boles, E. and Ciriacy, M. (1997), Eur. J. Biochem. 245: 324-333). In a yeast strain lacking the genes HXT1 through HXT7, growth on media containing high and low glucose concentrations (0.1% to 5%), glucose uptake and glucose consumption were below the detection level. In a series of experiments with mutant yeast strains expressing only one of the genes HXT1 through HXT7, it was shown that HXT11p and HXT3p are low-affinity transporters (km≈50-100 mM hexose), HXT4p is moderately low, and HXT2p, HXT6p and HXT7p are high affinity transporters (km≈1-4 mM hexose), regardless of the culture conditions of these mutants (0.1% or 5% glucose) (Reifenberger, E., Freidel, K. and Ciriacy, M. (1995) Yeast 11, S457). All hexose carriers display a stronger affinity for glucose compared to fructose. This is especially the case for the low affinity carriers HXT1 (km≈110 mM for glucose versus >300 mM for fructose) and HXT3 (km≈65 mM for glucose versus 125 mM for fructose).

The role of the HXT carriers has also been characterized during wine fermentation (Luyten, K., Riou, C. and Blondin, B. (2002) Yeast 19, 1-15 and Riou, C. Luyten, K. de Chazal, E. and Blondin, B. (2001) Yeast 18, S293). It was shown that under enological fermentation conditions several carriers (HXT1, HXT3, HXT6, HXT7) were involved in the hexose transport.

Following consumer demands and international winemaking practices, a large percentage of quality natural wines are fermented to dryness. This means that the amount of residual hexoses in the wines is usually below 1 g/L. Fructose is the main sugar present at the end of fermentations because fructose is more difficult to ferment than glucose, and therefore fermentations are often slow at the end. Depending on yeast activity, this can lead to sluggish or stuck fermentations. Yeasts that have a strong capacity to ferment fructose are expected to yield more rapid fermentation ends.

Yeast strains that are better able to ferment fructose have been isolated from nature and such so-called fructophilic yeasts have been successfully used to further reduce the sugar content of fermented must. They have also been successfully employed in stuck fermentations to eliminate the remaining sugar by inoculation with new yeast cells. A fructophilic yeast strain 67J INRA Narbonne called Fermichamp® has been isolated by the Institut National de Recherche Agronomique in France and made commercially available by DSM Food Specialties.

Next to fermentation of grapes for wine making, there are other industrial processes in which the fermentation rate and/or efficiency can be improved in order to increase the amount of alcohol produced. Examples of such processes are the production of fuel ethanol by fermentation of glucose derived from starch and the fermentation of xylose derived from xylan-containing compounds.

There is a need for yeast strains having improved carbohydrate utilisation capacity.

Surprisingly, it has now been found that the ability to better utilize carbohydrates resides in the HXT3 transporter. The invention relates to an isolated HXT3 gene encoding a HXT3 hexose transporter or functional fragments thereof, with an improved capacity to transport carbohydrates. The carbohydrates can for example be hexoses (glucose, fructose, galactose, mannose) and pentoses (xylose, ribose, arabinose). Preferably an improved capacity to utilize at least one of the carbohydrates selected from the group consisting of fructose, glucose or xylose, more preferably consisting of fructose and xylose, is obtained. In case of wine making an improvement in fructose utilisation is preferred, as described above. An additional advantage is that the fermentation rate can be increased as well by the HXT3 hexose transporter according to the invention.

The improvement of transport of a carbohydrate of a certain type can be measured by comparing the ratio of two carbohydrates including the one desired to analyse during the fermentation of a yeast transformed with either the mutated or the wild-type HXT3 gene. The wild-type HXT3 gene preferably has nucleic acid sequence according to SEQ ID NO: 25, being identical to S288C HXT3 gene transporter as known from Saccharomyces cerevisiae Genome Database, Stanford.

The yeast transformed with the HXT3 genes to be used in the measurement can be any desired yeast strain, as long as both genes are transformed in the same species.

Suitable yeast strains are for example Saccharomyces cerevisae, S. uvarum, S. bayanus, S. pastorianus, S. paradoxus. The person skilled in the art will be able to select the yeast strain intended for the desired application. Preferably Saccharomyces cerevisae is used in the measurement.

In case of an isolated HXT3 transporter having an improved capacity to transport fructose, the improved capacity to transport fructose with respect to a wild type hexose transporter is preferably measured by comparing the glucose/fructose ratio during fermentation (under the same conditions as described in example 4).

The present invention also relates to a new nucleic acid sequence encoding for a HXT3 transporter having an amino acid sequence derived from SEQ ID NO: 26 and having at least one mutation at a position selected from the group consisting of Leu 207, Met 208, Ile 209, Thr 210, Leu 211, Gly 212. Preferably the mutation is selected from the group consisting of Leu 207, Met 208, Ile 209, Thr 210, Leu 211, more preferably selected from the group consisting of Met 208, Ile 209, Thr 210 and most preferably the mutation is positioned at at Ile 209.

Also additional mutations can be present. Preferred additional mutations that attribute to the improved phenotype are mutations at or around at least one of the positions in the group consisting of positions 324, 388, 389, 392, 414, 415, 449, 471 of a wild type HXT3 transporter. In case of a HXT3 transporter according to SEQ ID NO: 26, this group consists more specifically of Met 324, Leu 388, Tyr 389, Ile 392, Glu 414, Gly 415, Ile 449, Leu 471.

Preferably any of the following mutations are additionally present either alone or in combination: Met 324 Ile, Leu 388 Met, Tyr 389 Trp, Ile 392 Val, Glu 414 Gin, Gly 415 Asn, Ile 449 Val, Leu 471 Ile.

The terms “mutated” or “mutation” or “mutations” in this context mean that a nucleotide sequence of the nucleic acid encoding the HXT3 transporter is different in comparison to the wild type HXT3 sequence of the species concerned. Alternatively, the terms “mutated” or “mutation” or “mutations” may refer to an alteration in the nucleotide sequence of a nucleic acid encoding the HXT3 transporter in comparison to the sequence of the gene encoding the endogenous HXT3 transporter. Additionally, the terms “mutated” or “mutation” or “mutations” are used herein to indicate alterations in the amino acid sequence of the hexose transporters in comparison to the natural or endogenous or wild type amino acid sequence.

The mutations may be conservative or non-conservative mutations.

The term “conservative substitution” is intended to mean a substitution in which the wild type amino acid residue is replaced with an amino acid residue having a similar side chain. These families are known in the art and include amino acids with basic side chains (e.g. lysine, arginine and histidine), acidic side chains (e.g. aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “non-conservative substitution” is intended to mean a substitution in which the wild type amino acid residue is replaced with an amino acid residue having a different side chain.

Surprisingly, it was found that even a conservative mutation as Ile 209 Val can contribute to the fructophilic phenotype, wherein the first three characters indicate the amino acid of the wild type HXT3 protein, the three digits in the middle represent the position of the mutation in the protein (Start Met=amino acid number 001) and the last three characters represent the amino acid of the mutated HXT3 protein according to the invention.

A number of specific mutations within the HXT3 gene is above identified that individually or in combination may attribute to the improved carbohydrate utilizing ability of yeasts. A number of specific mutations within the HXT3 gene is above identified that individually or in combination may attribute to the improved fructose utilizing ability of yeasts. It has furthermore been found that this mutated HXT3 gene can be transferred to a non-fructophilic strain and thereby improves the capacity to utilise fructose of this non-fructophilic strain during fermentation. Consequently, the invention relates to an isolated HXT3 gene comprising one or more mutations that improves the capacity of the gene product to transport fructose. The invention also relates to the specific gene and gene products derived there from as identified herein, as well as to yeast strains comprising a foreign mutated HXT3 gene.

In this context the term “foreign” refers to a gene that does not naturally occur in the genome of an organism, but instead has been acquired by the yeast through a recombination event, a mutation event or otherwise, such as by natural selection or by breeding.

In view of the subject-matter as disclosed in the present invention it would now be well within the skills of a person skilled in the art to find equivalents that work equally well but differ somewhat in the exact number and position of the mutations. These mutants may then be tested for their ability to utilize fructose as described in the examples and advantageous recombinants may easily be selected.

Alternatively, several mutation methods, which are known in the art, may be employed to introduce mutations in the HXT3 gene of any given yeast strain in order to render the strain more fructophilic. Examples of suitable yeast strains are Saccharomyces cerevisae, S. uvarum, S. bayanus, S. pastorianus, S. paradoxus. Also other genus of yeast can be transformed with the HXT3 gene according to the invention in order to increase its carbohydrate fermentation capacity for example Candida.

Also, the skilled person may find that mutations adjacent to the positions described above may yield useful recombinants. Any of the mutant HXT3 genes mentioned above are therefore encompassed in the present invention.

Surprisingly, the HXT3 gene plays a key role in fructose fermentation.

The mutations according to the invention allow the engineering of a number of HXT3 carriers in order to improve the capacity to utilise fructose in any given yeast strain.

The invention further relates to a process for obtaining a yeast cell with improved fructophilic properties wherein a yeast cell comprising a gene encoding an HXT3 transporter has been altered in such a way that the HXT3 transporter has an improved capacity to transport fructose, comprising the steps of:

a) mutating the HXT3 gene

b) selecting the yeast cell with improved fructophilic properties.

Altering the gene encoding an HXT3 transporter can be performed either via mutation or recombinant technology as known to the person skilled in the art. Also combinations hereof are possible, by, for example, first transforming the gene and then perform mutagenesis or point mutation on the transformed part. The person skilled in the art knows how to perform such mutations. Mutagenesis is for example described in WO04/070022 and it can be performed in an analogous way for Saccharomyces. The gene encoding an HXT3 transporter can be native or recombinant.

The mutated HXT3 gene can also be overexpressed, both in any given yeast strain or in Fermichamp®) itself. Overexpression of this gene triggers a higher fermentation rate than overexpression of a “standard” gene. This shows that the mutated protein is more efficient when over expressed. This allows to improve the fructose utlisation of other yeasts by transfer of the mutated HXT3 gene, as is effectively demonstrated in the examples. This can be of high interest in enology since fructose utilisation is one of the limiting factors of the fermentation rate at the end of the fermentation.

Furthermore, it has been found that in case the HXT3 transporter is overexpressed, the fermentation-glycolytic flux is enhanced. The differences in fermentation capacity triggered by overexpression of the two genes is therefore not limited to fructose transport capacity but also important for the fermentation of other carbohydrates. This may find also application in many fields where high fermentation rates are desirable, such as alcohol production and baking.

The invention further relates to the use of yeast according to the invention for fermentation of carbohydrates, more preferably fructose and glucose. The invention also relates to fermentation products produced by the strains according to the invention, for example alcohol, wine, beer, sake, vodka, ginever, tequila.

### EXAMPLES

### Example 1

### Strains and Culture Conditions

The S. cerevisiae strains used in this study are listed in table 1.

TABLE 1
Saccharomyces cerevisiae strains

| Strain | Genotype |
|---|---|
| Fermichamp © | Industrial strain |
| V5 | MATa ura3 gal |
| V5 HXT1-7Δ | V5 hxt514Δ::loxP |
| | hxt367Δ::loxP |
| | hxt2Δ::loxP |
| V5 HXT1-7ΔHXT3 (V5) | V5 hxt514Δ::loxP |
| | hxt367Δ::HXT3 from V5 hxt2Δ::loxP |
| V5 HXT1-7ΔHXT3 (Fermichamp) | V5 hxt514Δ::loxP |
| | hxt367Δ::HXT3 from Fermichamp |
| | hxt2Δ::loxP |
| V5 HXT1-7Δ + pHXT3 (V5) | V5 hxt514Δ::loxP |
| | hxt367Δ::loxP |
| | hxt2Δ::loxP + p4H7-HXT3V5 |
| V5 HXT1-7Δ + pHXT3 | V5 hxt514Δ::loxP |
| (Fermichamp) | hxt367Δ::loxP |
| | hxt2Δ::loxP + p4H7- |
| | HXT3Fermichamp |

Strain V5 is derived from the Champagne wine strain 8130. This strain was obtained by sporulation of the 8130 strain and the subsequent isolation of an ura3 mutant resistant to 5-fluoro orotic acid.

V5 HXT1-7Δ: This strain is deleted for HXT3-6-7 (deletion bounds localized from 1 164 600 to 1 154 055 on the chromosome IV) (positions according to Saccharomyces cerevisiae Genome Database, Stanford) and HXT5-1-4 (deletion bounds localized from 296 399 to 287 180 on the chromosome VIII) clusters. The strain is also deleted for HXT2 (deletion localized from position 288 125 to 289 658 of the chromosome XIII) resulting in a complete deletion of HXT1 to 7 genes. This strain is unable to grow on glucose or fructose.

Yeast strains were grown at 28° C. on YPD medium (except yeast strains transformed with p4H7 plasmid) containing either 2% glucose or 2% maltose (V5 HXT1-7Δ). For assessing the growth phenotype of the different integration mutant strains, they were grown on synthetic medium (0,67% Yeast Nitrogen Base without amino acid, 25 mg/l uracile, 5% glucose). Yeast strains transformed with p4H7plasmid containing HXT3 transporter genes were grown on synthetic medium (see above, without uracile). Batch enological-like fermentation experiments was carried out on synthetic must (MS300) containing 100 g/l glucose, 100 g/l fructose and an extra 115 mg/l methionine and 25 mg/l uracile (not for transformed yeast strains). This medium contains about 430 mg/l assimilable nitrogen. Precultured cells were inoculated at a density of 106 cells/ml in fermentors with a working volume of 1,1 l, equipped with fermentation locks. Fermentations were carried out at 28° C. with permanent stirring (500 rpm). These conditions give fermentation kinetics similar to those of industrial scale winemaking.

### Example 2

### Integration of HXT3 Into the V5 HXT1-7Δ Strain

The HXT3 genes originating either from V5 or Fermichamp were reintroduced into the V5 HXT1-7 deletion strain by genomic integration at the site of the original localization of their respective cluster. HXT3 gene was amplified by PCR using primers HXT3P1 and I2HXT3. These PCR amplification products were used for genomic integration when transformed in yeast and allow integration of a single copy of a HXT3 gene behind its own promotor. Correct integration was verified by PCR using C1HXT3ORF and C2HXT3p.primers for HXT3.

All the primers are listed in table 2.

TABLE 2
Primers used for HXT3 integration in V5 HXT1-7

| | | |
|---|---|---|
| Primers | Sequence 5′ to 3′ | Localization |

| | | |
|---|---|---|
| HXT3P1 | GTGCGGGATccGAAGGCAATATC | −1128 |
| HXT3P2 | gatcggATCCATCATCACGTTCCTAG | 2096 |
| | C | |
| I2HXT3 | aagtgacgggcgatgagtaagaaaga | 2095 |
| | aataactgactcattagaCCATCATC | |
| | ACGTTCCTAGC | |
| C1HXT3ORF | GACACAGTGACATATGCACC | 168 |
| C2HXT3p | TTAAGCATGATCGTCTAGGC | −1689 |
| HXT3p426 | aacacaaaaacaaaaagtttttttaa | −15 |
| | ttttaatcaaaaaCTGAGTTAAACAA | |
| | TCATGAATTCAACTCC | |
| HXT3t426 | gaatgtaagcgtgacataactaatta | 1694 |
| | catgactcgagACGGTTTAGCGTGAA | |
| | ATTATTTCTTGCC | |
| C′2HXT7p426 | Gccaatacttcacaatgttcg | −125 |

Underlined; homology to HXT7 terminator, UPPER CASE; homology to HXT3, Double underlined; homology to p4H7 promoter, Italics; homology to p4H7 terminator, Bold; homology to HXT7 promoter.

### Example 3

### Construction of p4H7 Multicopy Plasmid Containing HXT3 ORF

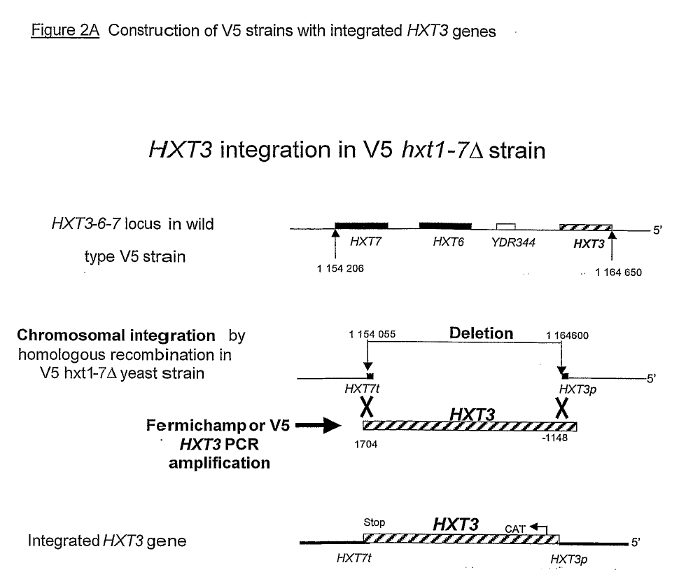

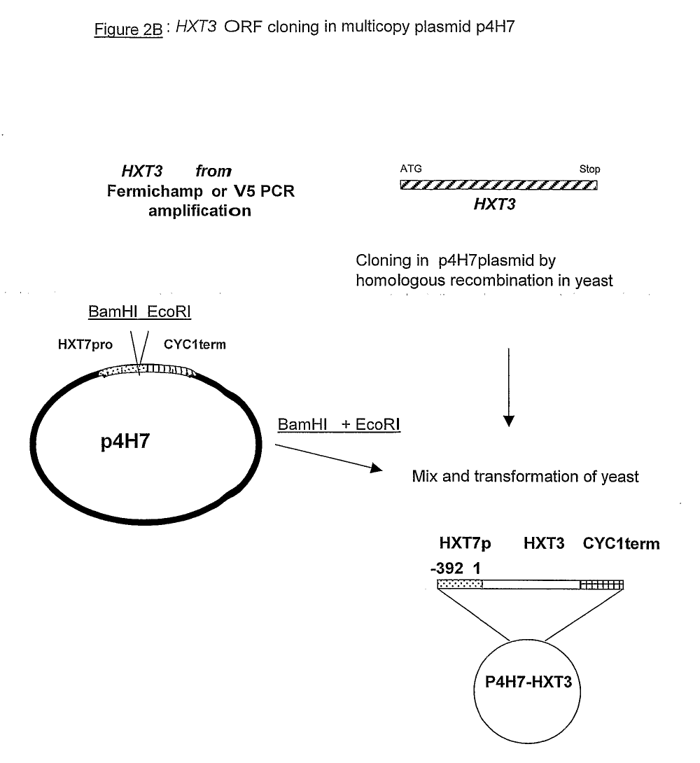

HXT3 genes were amplified by PCR from genomic DNA of V5 or Fermichamp strains using primers HXT3p426 and HXT3t426. The HXT3 genes were cloned in the plasmid p4H7 described in Hamacher et al., 2002. Microbiology, vol 148, 2783-2788, by in vivo recombination in Sacharomyces cerevisiae. The p4H7plasmid possess a truncated HXT7 promoter and a CYC1 terminator. The p4H7plasmid was first linearized with BamH1 & EcoR1. The 5′ end of primer HXT3p426 is homologous to the BamH1 end (HXT7 promoter) of the p4H7 plasmid linearized with BamH1 & EcoR1. The 5′ end of primer HXT3t426 is homologous to the EcoR1 end (terminator side) of the p4H7plasmid linearized with BamH1 & EcoR1. PCR amplification products for HXT3 and the p4H7 plasmid linearized with EcoR1 and BamH1 were used to yeast as depicted in FIG. 2. Transformants were selected for their ability to grow on a minimal medium, which contained glucose as sole carbon source. The resulting recombined plasmids have the HXT3 ORF behind the truncated and unregulated HXT7 promoter leading to overexpression of HXT3. All the primers are listed in table 2.

### Example 4

### Analytical Methods

Monitoring of Fermentation

CO2 release was determined by automatic measurement of fermentor weight loss for 20 min each. The CO2 production rate was automatically calculated by polynomial smoothing of the CO2 evolved. This method of fermentation monitoring provides high reproducibility. The measure of the total CO2 evolved was used to check the completion of sugar fermentation. Experiments were done at least in duplicate, representative results are shown.

Monitoring of Glucose and Fructose Consommation

During fermentation, medium is taken at least two times a day, centrifuged to remove cells and supernatant is stored at −20° C. before using for glucose and fructose measurement by HPLC. Sugars were analysed by HPLC using a Hewlett-Packard HP series 1100 system equipped with an Aminex 87H column (Bio-Rad Laboratories). Fermentation supernatants were diluted ⅙th in the mobile phase (0.004 M H₂SO₄) and sugars were detected by refractometry.

### Example 5

### Sequence Analysis of HXT3 Genes of the Fermichamp Strain

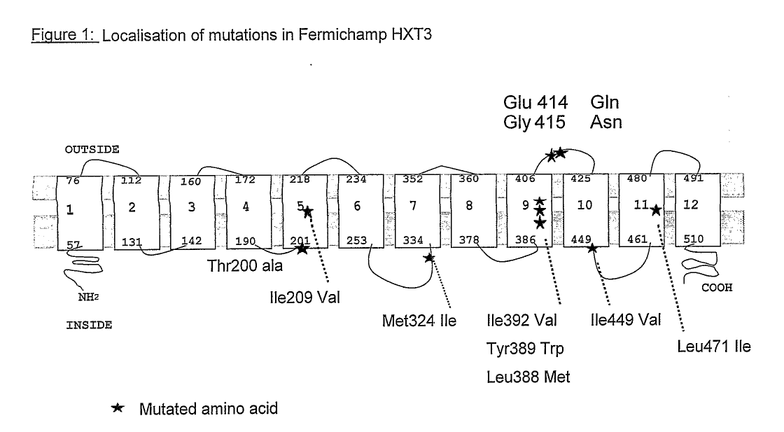

HXT3 gene was amplified by PCR using primers HXT3P2 and HXT3P1 (primers are shown in table 2.). After purification, PCR products were sequenced and the results are shown in tables 3A and B. The promoter region of the HXT3 gene (nucleotides −900 to 1) displays only 6 mutations while the coding region (nucleotides 1 to 1700) harbours 38 mutations. Ten of these mutations lead to amino acid changes in the protein sequence when compared with the sequence of the glucophilic wild type strain S288C. Most of these changes are clustered in a region of the protein that includes one membrane spanning domain and an external loop (FIG. 1). Most of the changes are conservative substitutions. The HXT3 gene from strain V5 appeared to be identical to that of S288C (Saccharomyces cerevisiae Genome Database, Stanford).

TABLE 3A
Fructophilic mutations in the HXT3 gene (promotor) from
Fermichamp ® in comparison with S288C (and V5).
Promoter (−900 − 1)

−859 C → T
−602 A → T
−439 T → deletion
−282 A → T
−278 T → C
−88 C → T

TABLE 3B
Fructophilic mutations in the HXT3 gene (open reading frame) from
Fermichamp ® in comparison with S288C (and V5).

| ORF (1-1700) | Amino acids (1-567) |
|---|---|
| 598 A → G | 200 Thr → Ala |
| 625 A → G | 209 Ile → Val |
| 972 G → A | 324 Met → Ile |
| 1162 T → A | 388 Leu → Met |
| 1164 A → G | 388 |
| 1166 A → G | 389 Tyr → Trp |
| 1167 T → G | 389 |
| 1174 A → G | 392 Ile → Val |
| 1176 T → C | 392 |
| 1240 G → C | 414 Glu → Gln |
| 1243 G → A | 415 Gly → Asn |
| 1244 G → A | 415 |
| 1245 T → C | 415 |
| 1445 A → G | 449 Ile → Val |
| 1411 T → A | 471 Leu → Ile |
| 1413 G → C | 471 |

### Example 6

### Expression of HXT3 Integrated in V5HXT1-7ΔStrain

The HXT3 genes originating either from V5 or Fermichamp were introduced into the V5 HXT1-7Δ strain by integration as indicated in material and methods. Details of the positions of integration at the HTX3 loci are given in FIG. 2. After yeast transformation with the PCR products containing the HXT3 gene, the transformants were directly selected on a medium containing only glucose as carbon source.

The V5 HXT1-7D strains containing the integrated HXT3 gene originating from V5 or from Fermichamp were obtained and termed V5 HXT1-7ΔHXT3 (V5) and V5 HXT1-7ΔHXT3 (Fermichamp) respectively.

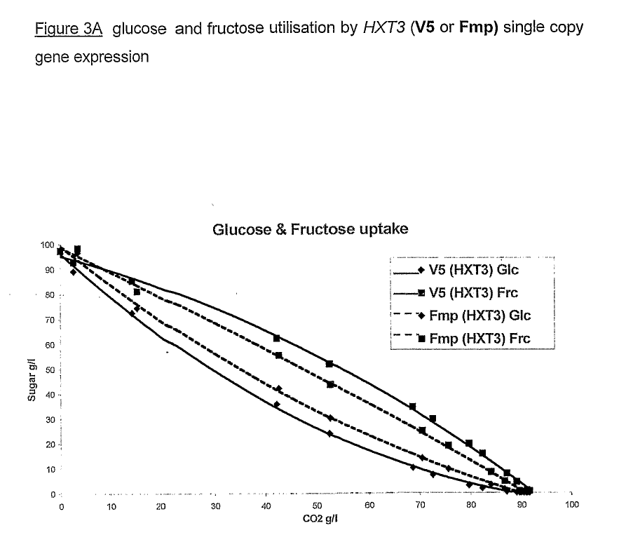

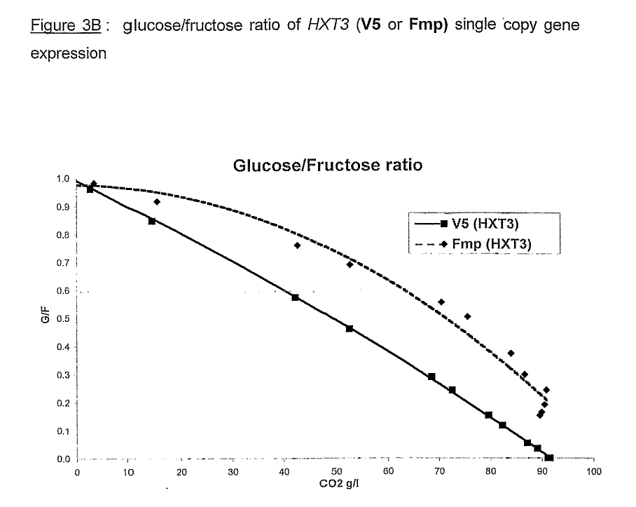

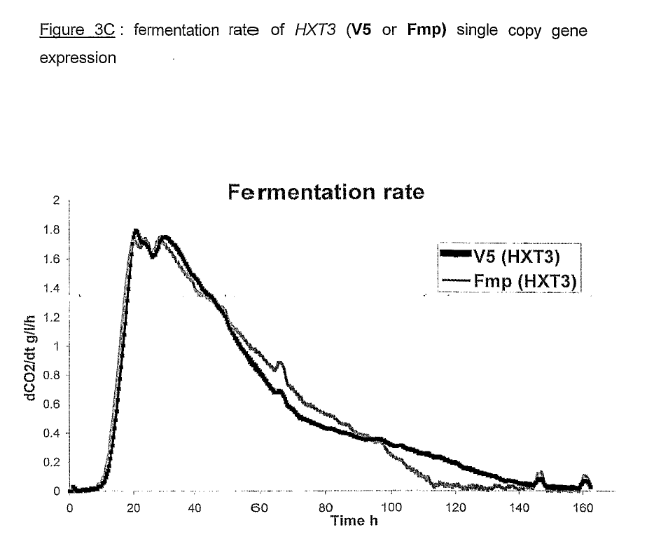

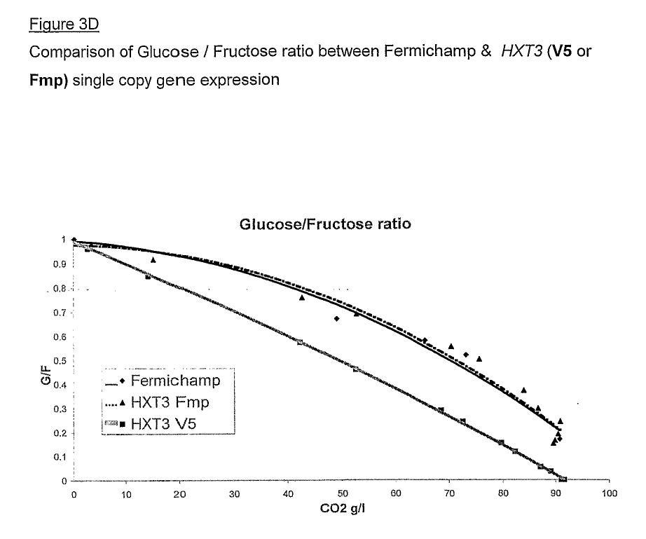

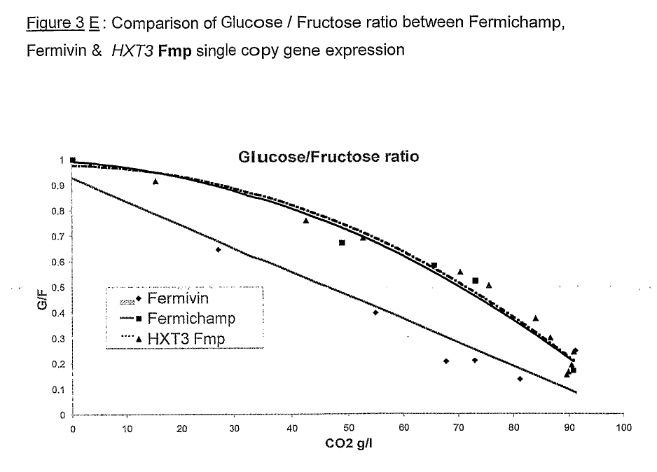

The resulting strains were examined for their fermentation properties, fermentation rate and glucose/fructose utilisation. The V5 HXT1-7ΔHXT3 (V5) and V5 HXT1-7ΔHXT3 (Fermichamp) displayed different profiles of sugar utilisation (FIGS. 3a, b). The relative rate of fructose and glucose utilisation differ and the strain expressing the HXT3 gene from Fermichamp displays a higher capacity to use fructose. The ratio glucose/fructose is maintained at higher levels with the strain expressing the HXT3 gene from Fermichamp.

Comparison of the evolution of the ratios during the fermentation shows that the strain expressing the HXT3 gene from Fermichamp exhibits a profile similar to the Fermichamp strain while the one expressing the HXT3 gene from V5 displays a “standard” glucose/fructose profile, similar to Fermivin (FIGS. 3d, e). The Fermichamp fructose utilisation capacity is therefore triggered in the V5 HXT1-7Δ strain by expression of the Fermichamp HXT3 gene.

The fermentation rate profiles are also significantly influenced by the HXT3 carrier expressed. Although no differences are observed in the first part of the fermentation, a higher fermentation rate is obtained at the end of the fermentation when the gene from Fermichamp is expressed (FIG. 3c). Consistently, the fermentation time is reduced in recombinants carrying this gene. The better fermentation rate at the end of the fermentation is in agreement with a better capacity to use fructose at the end (which is the main sugar present) and to a minor glucose/fructose disequilibrium in this late phase.

### Example 7

### Effect of HXT3 Gene Overexpression

The HXT3 genes from Fermichamp and from V5 were over expressed in V5 HXT1-7Δ. The HXT3 gene from Fermichamp or V5 was introduced on a multicopy plasmid, which allows an unregulated and high expression of the corresponding gene (see material and methods).

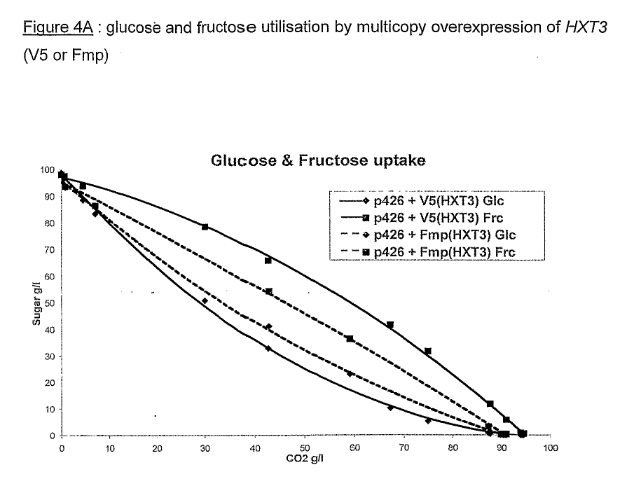

As shown in FIG. 4, the overexpression of the HXT3 genes does not significantly modify the fructose/glucose utilisation of the strain compared to the integrated, single copy, strain. A slight enhancement of the fructose utilisation improvement is however observed.

The overexpression of the HXT3 genes from V5 and Fermichamp triggers very different effects on the fermentation rate on a MS300 medium containing glucose and fructose (50/50). Overexpression of the HXT3 gene from V5 has only a little effect on the fermentation rate compared to the integrated single copy (FIG. 5). Overexpression of the HXT3 gene from Fermichamp triggers a strong improvement of the fermentation rate and an important reduction of the fermentation time. The fermentation rate obtained by overexpression of the HXT3 gene from Fermichamp is much higher than that obtained with the gene from V5.

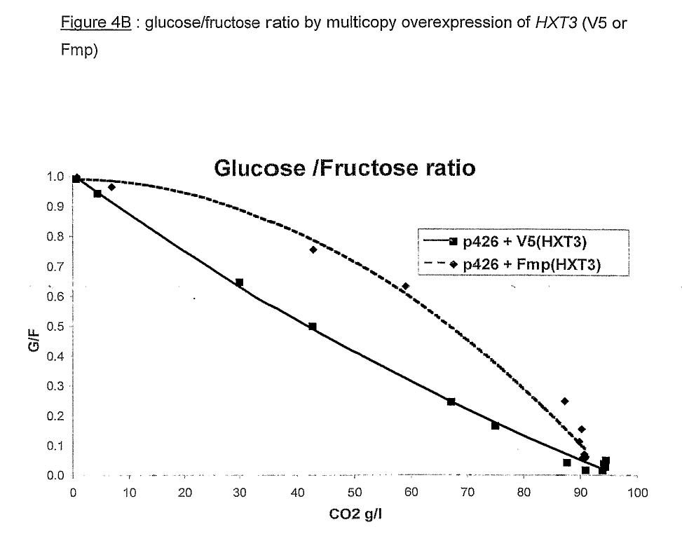

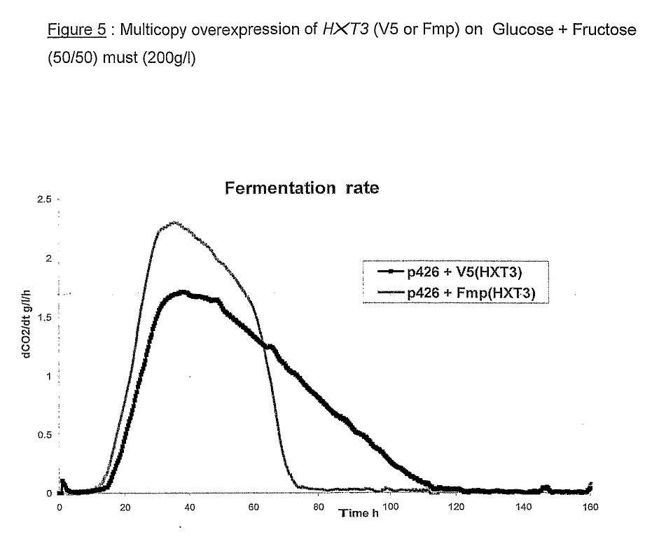

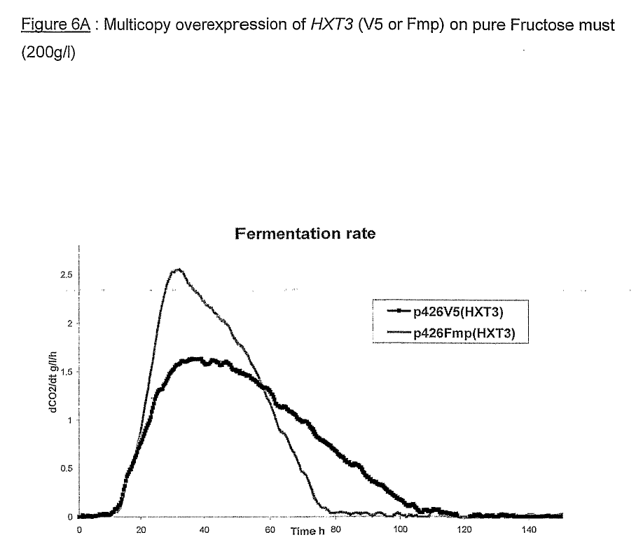

In order to investigate whether the effect of the overexpression on the fermentation rate was due to a better utilisation of fructose or not, we have examined the fermentation capacity of the HXT3 overexpressing strains in a MS medium containing only glucose or only fructose. As shown FIGS. 6a, b, the overexpression of HXT3 from Fermichamp triggers a much higher fermentation rate than the overexpression of the gene from V5, independently of the sugar fermented. The strong improvement of the fermentation capacity compared to the strain expressing the V5 gene, is observed with glucose as well as with fructose. The differences in fermentation capacity triggered by overexpression of the two genes is therefore independent of their fructose transport capacity.

Single copy expression of the HXT3 genes (in the integrated strains) does not lead to the same picture on pure sugar fermentations (FIGS. 7a, b). The HXT3 gene from Fermichamp gives a significant improvement of the fermentation end when only fructose is in the medium. Only a slight difference in fermentation profile is observed between the two genes when glucose is the sugar fermented. This indicates to that when the expression of the HXT3 gene is low, the difference in fermentation rate is mainly due to the improved capacity to transport fructose.

### Example 8

### Assessment of the Role of Various Mutations on the Fructose Fermentation Phenotype.

### Construction of Chimeric HXT3 Proteins

The contribution of various group of mutation in the Fermichamp HXT3 protein was addressed by constructing strains that express chimeric proteins that contain part(s) of HXT3 sequence originating from Fermichamp and other part(s) from a standard (V5) HXT3 sequence.

### Construction of Strains That Contain a Single, Inactive, HXT3 Gene

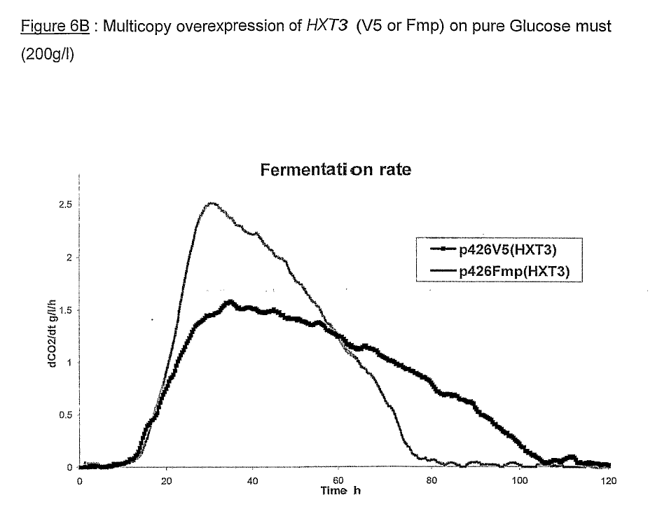

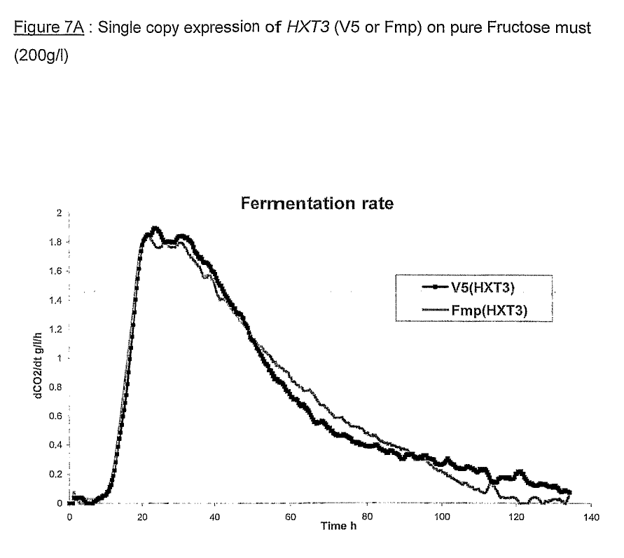

In order to create such chimera several yeast strains with a disrupted HXT3 copy were constructed. These strains were created by insertion of the KanMX cassette in the HXT3 sequence of one of the strain containing a single HXT3 gene coming from either Fermichamp or from V5. The principle of such strain construction is presented FIG. 8.
The strain V5HXT1-7ΔHXT3 (Fermichamp) Δ KanMX571-650 was created by transformation of the strain V5HXT1-7ΔHXT3 (Fermichamp) with a PCR DNA product that contained the KanMX gene flanked by HXT3 Fermichamp sequence. This DNA fragment was obtained by PCR amplification using the PUG6 plasmid that carries the KanMX gene (Güldener et al., 1996. Nucleic Acids Res. 24, 2519-2524) as DNA matrix encoded for part by the standard (V5) HXT3 sequence and for other part by the HXT3 Fermichamp sequence. The expression of these a chimeric HXT3 proteins restores the growth of the transformed yeasts on glucose and are selected for growth on YPD (YP-glucose 2%).

### Strain Expressing Chimeric HXT3 Proteins

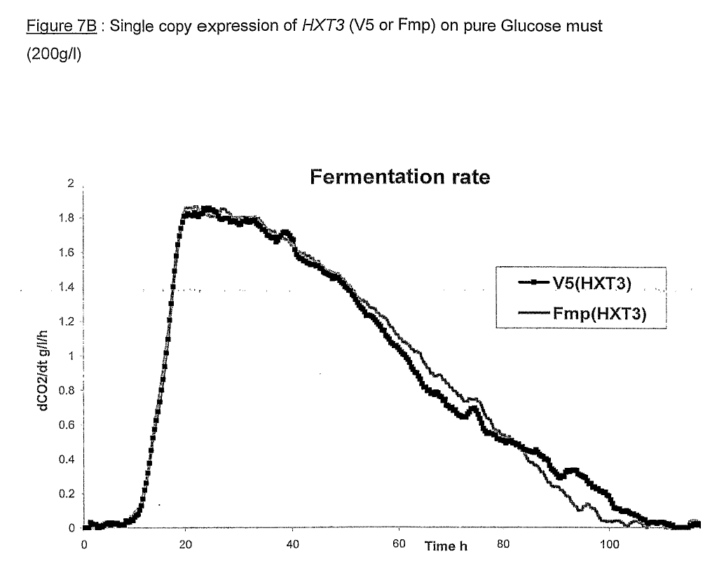

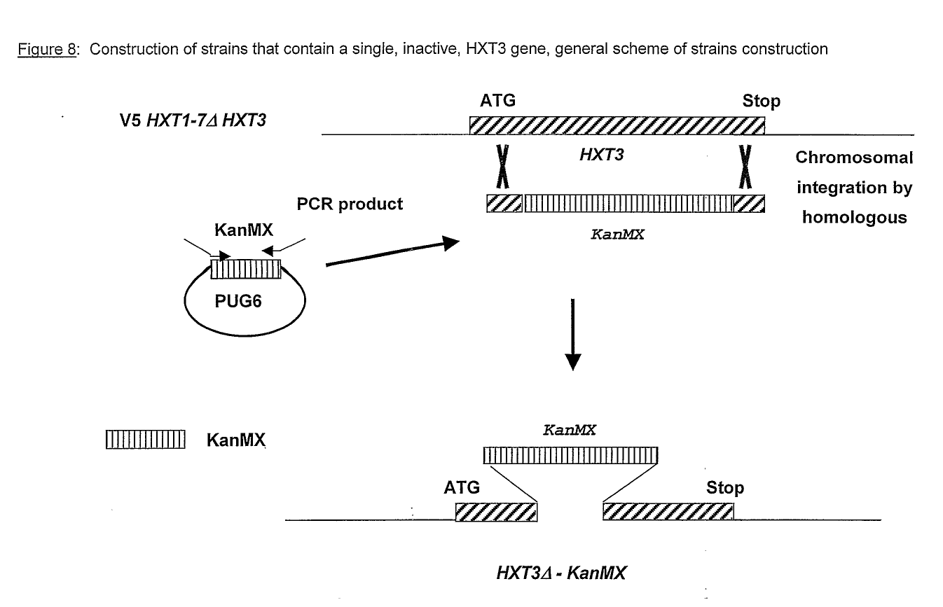

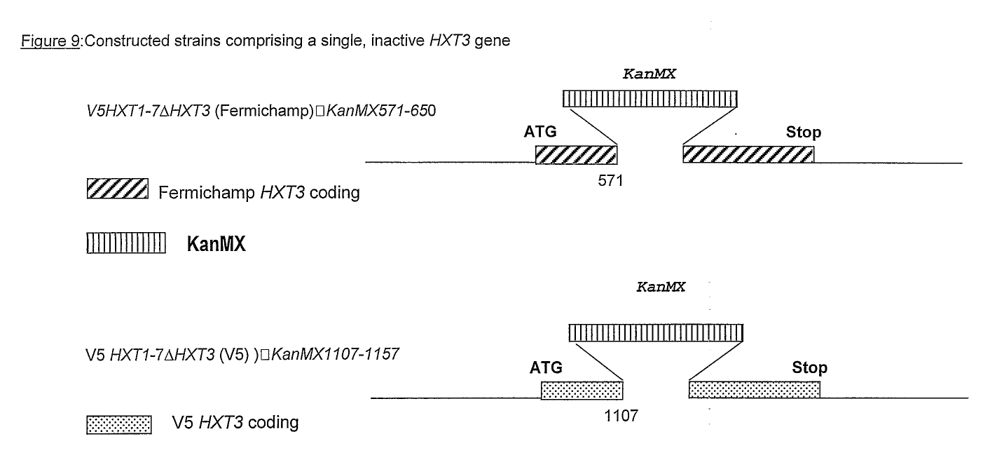

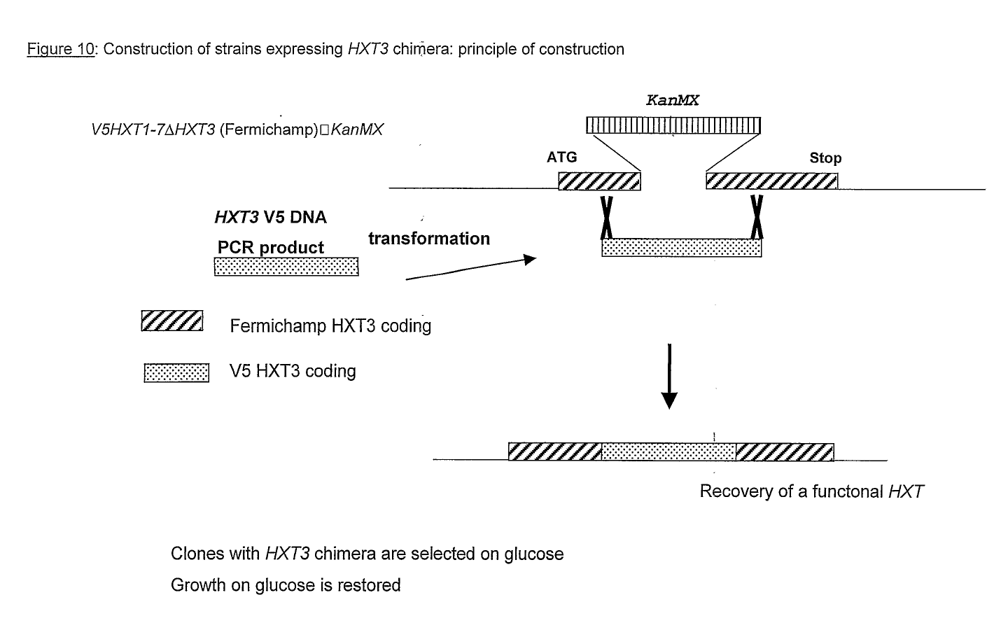

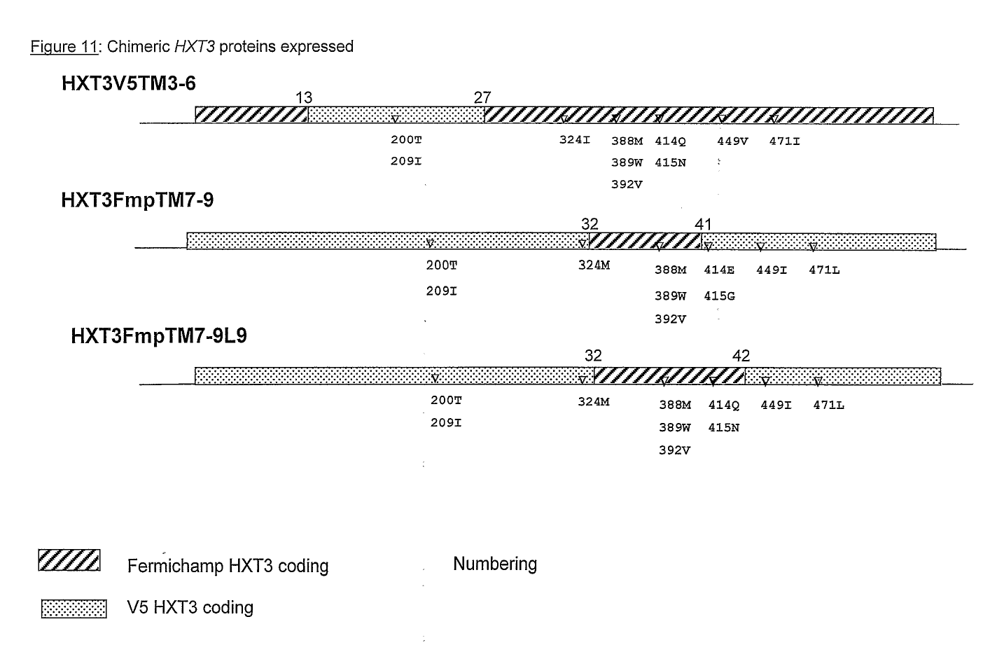

The chimeric proteins expressed are presented in FIG. 11.

A strain expressing the chimera HXT3V5TM3-6 was obtained by amplifying an HXT3 fragment, coordinates 397 to 818, using primers IA397-416 and IA818-798 (Table 5),from the V5 strain DNA and transformation of the strain V5HXT1-7ΔHXT3 (Fermichamp) ΔKanMX571-650 with the PCR product. Transformants were selected on the YPD medium. The resulting strain express an HXT3 protein which sequence is encoded by Fermichamp HXT3 gene except from nucleotides 397 to 818 (aa 132 to 272) encoded by V5 HXT3 sequence. This corresponds to the replacement of the two mutated amino acids A200 and V209 of Fermichamp HXT3 by the standard amino acids T200 and I209.

A strain expressing the chimera HXT3FmpTM7-9 was obtained by PCR amplification of an HXT3 fragment, coordinates 973 to 1232, using primers IIIC973-992 and IIIC1232-1213 (Table 5), from Fermichamp DNA and transformation of the strain V5 HXT1-7ΔHXT3 (V5) ΔKanMX1107-1157. Transformants were selected on the medium YPD. The resulting strain express an HXT3 protein which sequence is encoded by V5 HXT3 gene except from nucleotides 973 to 1232 (aa 325 to 410) which is encoded by Fermichamp HXT3 sequence. This corresponds to the introduction of 3 mutated amino acids of Fermichamp M388, W389, V392 in the standard HXT3 sequence.

A strain expressing the chimera HXT3FmpTM7-9L9 was obtained by PCR amplification of an HXT3 fragment, coordinates 973 to 1280, using primers IIID 973-992 and IIID1 1280-1261 (Table 5), from the Fermichamp DNA and transformation of the strain V5 HXT1-7ΔHXT3 (V5) ΔKanMX1107-1157. Transformants were selected on the medium YPD. The resulting strain express an HXT3 protein which sequence is encoded by V5 HXT3 gene except from nucleotides 973 to 1280 (aa 325 to 427) which is encoded by Fermichamp HXT3 sequence. This corresponds to the introduction of 5 mutated amino acids of Fermichamp M388, W389, V392, Q414, N415 in the standard HXT3 protein sequence.

TABLE 5
Primers used to amplify HXT3 DNA

| | |
|---|---|
| Primers | Sequence 5′ to 3′ |

| | |
|---|---|
| IA 397-416 | TTGGGTGATATGTACGGTCG |
| IA 818-798 | AGAGATGCTCTTGGTTCGTC |
| IIIC 973-992 | GGTATCATGATCCAATCTCT |
| IIIC 1232-1213 | GGCCATAATCTAGTGACTCC |
| IIID 973-992 | GGTATCATGATCCAATCTCT |
| IIID1 1280-1261 | ATCATaCAGTTACCAGCAcc |

### Switching of Amino Acids by Site Directed Mutagenesis

Cloning of the HXT3 Gene from Fermichamp in the pUC19 Plasmid
The HXT3 coding DNA was PCR amplified from genomic Fermichamp DNA using the primers BamHXT3ATG_F and HindHXT3STOP_R (Table 6). These primers allowed the amplification of the complete ORF and added a BamHI restriction site at the 5′ end and a HindIII site at the 3′ end of HXT3 sequence. The pUC19 DNA and the amplified HXT3 Fermichamp DNA were digested with Bam HI and HindIII restriction enzymes, purified and used for ligation. The ligation mixture was used to transform E. coli, DH5a.

### Site Directed Mutagenesis

Recombinant PUC19 plasmid DNA carrying the HXT3 gene was used for site directed mutagenesis. Site directed mutagenesis was performed using the Stratagene QuikChange™ site directed mutagenesis kit based on the use of two complementary oligonucleotide primers containing the desired mutation for the amplification with a PfuTurbo™ DNA polymerase.

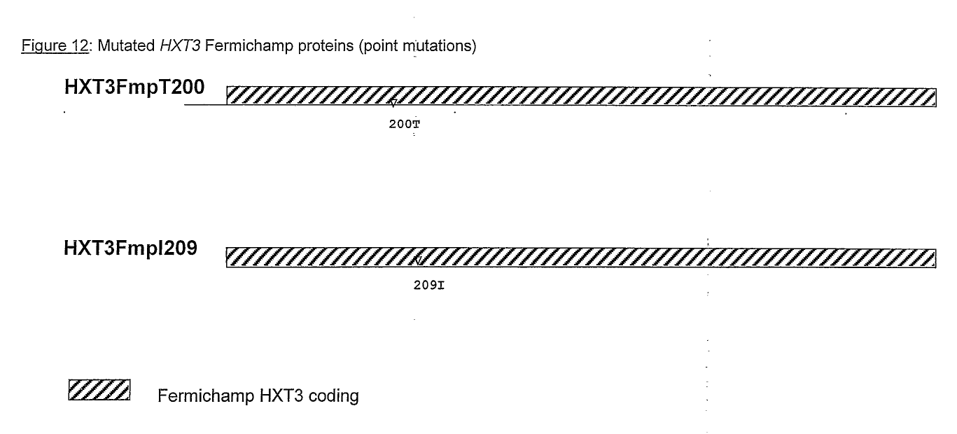

The couple of oligonucleotides FmpT200-F and FmpT200-R (Table 6) was used for site directed mutagenesis to create the construct HXT3FmpT200. This led to the replacement of the A200 of the Fermichamp HXT3 sequence by the standard amino acid T200 of HXT3 (FIG. 12).

The couple of oligonucleotides FmpI209-F and FmpI209-R (table 6) was used for site directed mutagenesis to create the construct HXT3FmpI209. This led to the replacement of the V209 of the Fermichamp HXT3 sequence by the standard amino acid I209 of HXT3 (FIG. 12).

The mutated HXT3 genes were PCR amplified using the primers IA 397-416 and IA 818-798 (Table 5), the PCR product was used for transformation of the strain V5HXT1-7ΔHXT3 (Fermichamp) ΔKanMX571-650. The transformed strains were selected on a YPD medium. Two strains were obtained. One expressed the construct HXT3FmpT200 and is designed as V5HXT1-7Δ-HXT3FmpT200. The other expressed the construct HXT3FmpI209 and is designed as V5HXT1-7Δ-HXT3FmpI209.

TABLE 6

Primers used for cloning HXT3 in pUC19
and for point mutagenesis

| | |
|---|---|
| Primers | Sequence 5′ to 3′ |

| | |
|---|---|
| Bam HXT3ATG_F | CgaggggatccAATCATGAATTCAACTCCAG |
| Hind HXT3Stop_R | cgaggaagcttCGTGAAATTATTTCTTGCCG |
| FmpT200_F | CCTAAGGAAATGAGAGGTaCTTTAGTCTCCTGTT |
| | ACC |
| FmpT200_R | GGTAACAGGAGACTAAAGtACCTCTCATTTCCTT |
| | AGG |
| FmpI209_F | CCTGTTACCAACTGATGaTTACCTTGGGTATTTT |
| | CTTGGG |
| FmpI209_R | CCCAAGAAAATACCCAAGGTAAtCATCAGTTGGT |
| | AACAGG |

Homology to HXT3, upper case; restriction site, italic; introduced mutation for amino acid switch, bold

Analysis of Glucose-Fructose Utilisation Profiles During Alcoholic Fermentation with the Strains Expressing the Chimeric or Mutated HXT3 Carriers

The properties of the strains expressing the chimeric and mutated HXT3 proteins were assessed during alcoholic fermentation on the MS300 medium containing 100 g/L glucose and 100 g/L fructose. The sugar utilisation properties were examined and expressed as the glucose/fructose ratio evolution as a function of the fermentation progress.

The glucose-fructose utilisation profile of the strain expressing the chimera HXT3V5TM3-6 is shown FIG. 13. This strain displays a sugar utilisation profile identical to the V5 strain. This indicates that the one or both of the amino acids remove from Fermichamp protein, A200 or (and) V209 is (or are) essential for the fructose utilisation property given by Fermichamp HXT3. The removal of these two mutated amino acids from Fermichamp led to the loss of the fructose utilisation property.

The glucose-fructose utilisation profile of the strain expressing the chimera HXT3FmpTM7-9 is shown FIG. 14. This strain displays a sugar utilisation profile identical to the V5 strain. This indicates that the introduction of the 3 mutated amino acids M388, W389, V392 of Fermichamp are not sufficient to trigger the fructose utilisation property given by Fermichamp HXT3.

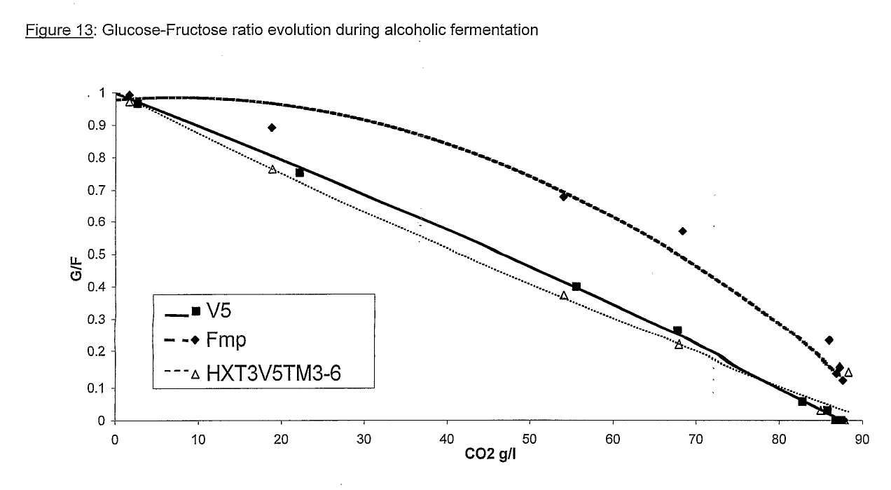

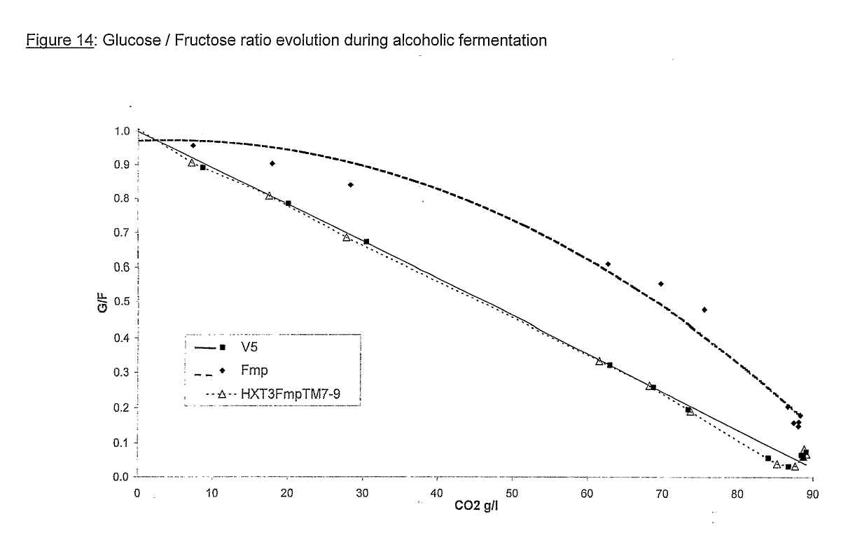

The glucose-fructose utilisation profile of the strain expressing the chimera HXT3FmpTM7-9L9 is shown FIG. 15. This strain displays a sugar utilisation profile identical to the V5 strain. This indicates that the 5 amino acids M388, W389, V392 Q414, N415 alone are not sufficient to trigger the fructose utilisation property given by Fermichamp HXT3.

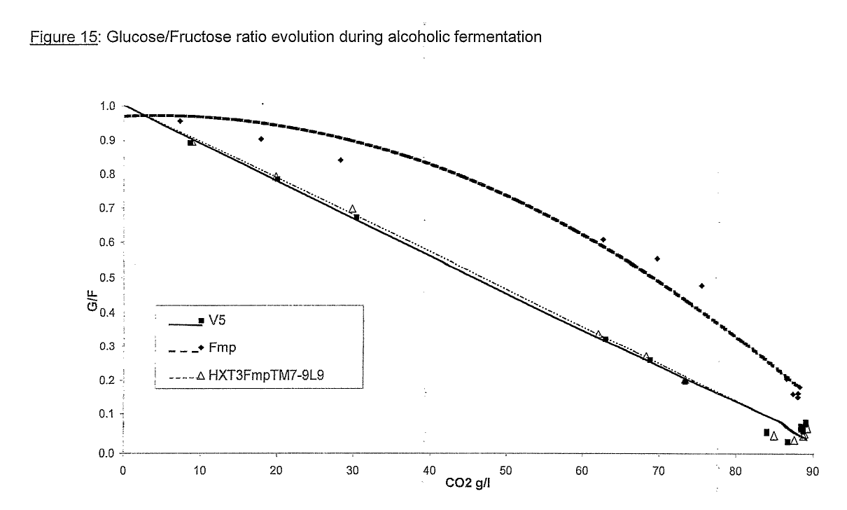

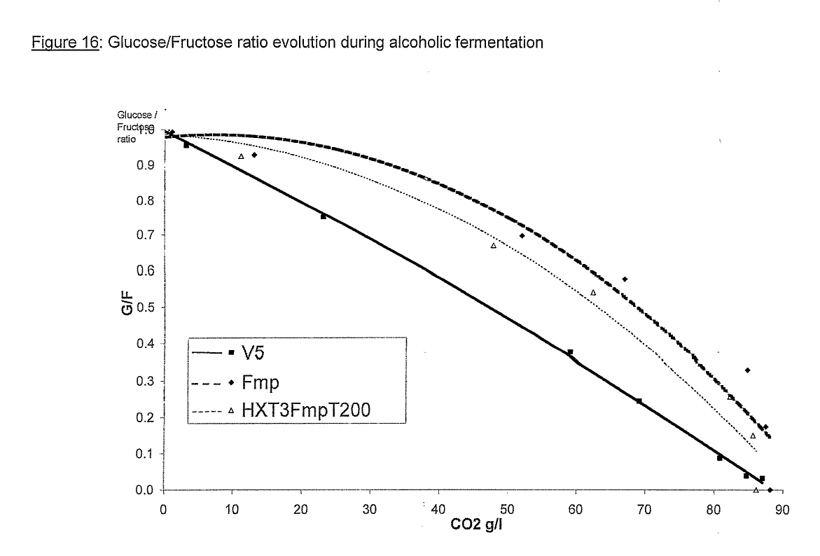

The glucose-fructose utilisation profile of the strain expressing the mutated Fermichamp carrier HXT3FmpT20O is shown FIG. 16. This strain displays a sugar utilisation profile identical to the Fermichamp strain. This indicates that the amino acid A200 is not essential for the fructose utilisation property given by Fermichamp HXT3.

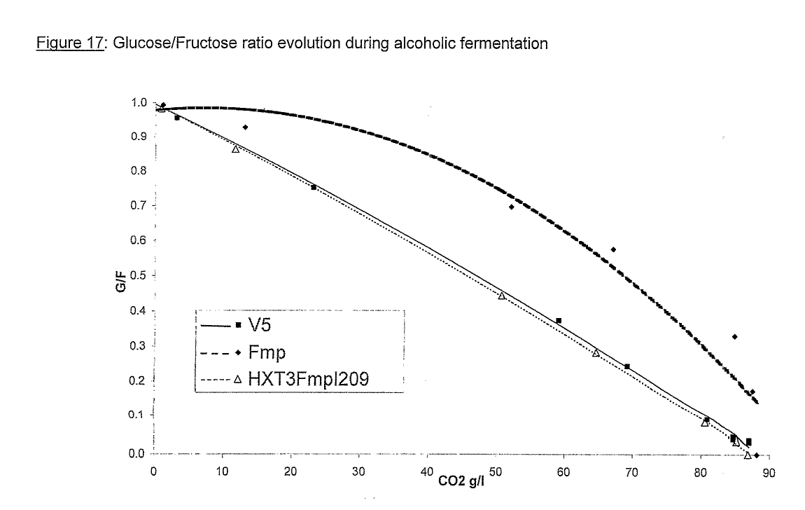

The glucose-fructose utilisation profile of the strain expressing the mutated Fermichamp carrier HXT3FmpI209 is shown in FIG. 17. This strain displays a sugar utilisation profile identical to the V5 strain. This indicates that the presence of amino acid Ile 209 is essential for the fructose utilisation property given by Fermichamp HXT3.

1. An isolated HXT3 hexose transporter or functional fragments thereof with an improved capacity to transport carbohydrates.

2. An isolated HXT3 hexose transporter having an improved capacity to transport fructose with respect to the capacity to transport fructose of a wild type hexose transporter having SEQ ID NO: 26.

3. The HXT3 hexose transporter according to claim 1, having an amino acid sequence selected from the group consisting of:
a sequence derived from SEQ ID NO: 26 and having at least a mutation at a position selected from the group consisting of Gln 206, Leu 207, Met 208, Ile 209, Thr 210, Leu 211 and Gly 212; or
SEQ ID NO: 27.

4. The HXT3 hexose transporter according to claim 3, additionally comprising at least a mutation at a position selected from the group consisting of Met 324, Leu 388, Ile 392, Glu 414, Gly 415, Ile 449 and Leu 471.

5. An isolated nucleic acid encoding the HXT3 hexose transporter according to claim 1.

6. The nucleic acid sequence according to claim 5, having a sequence according to SEQ ID NO: 28, SEQ ID NO: 29 or a functional homologue thereof.

7. Recombinant yeast cell transformed with a nucleic acid according to claim 5.

8. Process for obtaining a yeast cell with improved fructophilic properties wherein a yeast cell comprising a gene encoding an HXT3 transporter has been altered in such a way that the HXT3 transporter has an improved capacity to transport fructose, comprising the steps of:
a. mutating the HXT3 gene and
b. selecting the yeast cell with improved fructophilic properties.

9. Yeast cell obtainable by the process according to claim 8.

10. Yeast cell according to claim 7, wherein the yeast is Saccharomyces cerevisae, S. uvarum, S. bayanus, S. pastorianus or S. paradoxus.

11. Use of a yeast according to claim 9 for fermentation of carbohydrates.

12. The HXT3 hexose transporter according to claim 1, having an amino acid sequence which is a sequence derived from SEQ ID NO: 26 and having at least a mutation at a position at Ile 209.

13. The HXT3 hexose transporter according to claim 3, additionally comprising at least a mutation selected from the group consisting of Met 324 Ile, Leu 388 Met, Tyr 389 Trp, Ile 392 Val, Glu 414 Gln, Gly 415 Asn, Ile 449 Val and Leu 471 Ile.


SEQUENCE LISTING (30 sequences)

SEQ ID NO: 1 - DNA, 23 bp, Artificial Sequence
  primer
gtgcgggatccgaaggcaatatc

SEQ ID NO: 2 - DNA, 27 bp, Artificial Sequence
  primer
gatcggatccatcatcacgttcctagc

SEQ ID NO: 3 - DNA, 63 bp, Artificial Sequence
  primer
aagtgacgggcgatgagtaagaaagaaataactgactcattagaccatcatcacgttcctagc

SEQ ID NO: 4 - DNA, 20 bp, Artificial Sequence
  primer
ttaagcatgatcgtctaggc

SEQ ID NO: 5 - DNA, 68 bp, Artificial Sequence
  primer
aacacaaaaacaaaaagtttttttaattttaatcaaaaactgagttaaacaatcatgaattcaactcc

SEQ ID NO: 6 - DNA, 65 bp, Artificial Sequence
  primer
gaatgtaagcgtgacataactaattacatgactcgagacggtttagcgtgaaattatttcttgcc

SEQ ID NO: 7 - DNA, 20 bp, Artificial Sequence
  primer
gacacagtgacatatgcacc

SEQ ID NO: 8 - DNA, 21 bp, Artificial Sequence
  primer
gccaatacttcacaatgttcg

SEQ ID NO: 9 - DNA, 60 bp, Artificial Sequence
  primer
tgttggtggtattgccgttttatctcctatgttgatttctttcgtacgctgcaggtcgac

SEQ ID NO: 10 - DNA, 62 bp, Artificial Sequence
  primer
cacagagttggagtagttcttagtaccgaagttggtacaggcataggccactagtggatctg

SEQ ID NO: 11 - DNA, 60 bp, Artificial Sequence
  primer
tttcgaaacttctattgttttcggtgtcgtcaacttcttcttcgtacgctgcaggtcgac

SEQ ID NO: 12 - DNA, 62 bp, Artificial Sequence
  primer
acataacagcagaccataccaatggcaccatataacaaacgcataggccactagtggatctg

SEQ ID NO: 13 - DNA, 20 bp, Artificial Sequence
  primer
ttgggtgatatgtacggtcg

SEQ ID NO: 14 - DNA, 20 bp, Artificial Sequence
  primer
agagatgctcttgcttcgtc

SEQ ID NO: 15 - DNA, 20 bp, Artificial Sequence
  primer
ggtatcatgatccaatctct

SEQ ID NO: 16 - DNA, 20 bp, Artificial Sequence
  primer
ggccataatctagtgactcc

SEQ ID NO: 17 - DNA, 20 bp, Artificial Sequence
  primer
ggtatcatgatccaatctct

SEQ ID NO: 18 - DNA, 20 bp, Artificial Sequence
  primer
atcatacagttaccagcacc

SEQ ID NO: 19 - DNA, 31 bp, Artificial Sequence
  primer
cgaggggatccaatcatgaattcaactccag

SEQ ID NO: 20 - DNA, 31 bp, Artificial Sequence
  primer
cgaggaagcttcgtgaaattatttcttgccg

SEQ ID NO: 21 - DNA, 37 bp, Artificial Sequence
  primer
cctaaggaaatgagaggtactttagtctcctgttacc

SEQ ID NO: 22 - DNA, 37 bp, Artificial Sequence
  primer
ggtaacaggagactaaagtacctctcatttccttagg

SEQ ID NO: 23 - DNA, 40 bp, Artificial Sequence
  primer
cctgttaccaactgatgattaccttgggtattttcttggg

SEQ ID NO: 24 - DNA, 40 bp, Artificial Sequence
  primer
cccaagaaaatacccaaggtaatcatcagttggtaacagg

SEQ ID NO: 25 - DNA, 1704 bp, Saccharomyces cerevisiae
atgaattcaactccagatttaatatctccacaaaagtcaagtgagaattcgaatgctgacctgccttcgaatagctctcaggtaatgaacatgcctgaagaaaaaggtgttcaagatgatttccaagctgaggccgaccaagtacttaccaacccaaatacaggtaaaggtgcatatgtcactgtgtctatctgttgtgttatggttgccttcggtggtttcgttttcggttgggatactggtaccatttctggtttcgtcgcccaaactgatttcttgagaagattcggtatgaagcataaagatggtagttattatttgtctaaggttagaactggtttaattgtctccattttcaacattggttgtgccattggtggtattattttggctaaattgggtgatatgtacggtcgtaaaatgggtttgattgtcgttgttgttatctacatcatcggtattattattcaaattgcatccatcaacaaatggtaccaatatttcatcggtagaattatttccggtttgggtgttggtggtattgccgttttatctcctatgttgatttctgaagtcgctcctaaggaaatgagaggtactttagtctcctgttaccaactgatgattaccttgggtattttcttgggttactgtaccaacttcggtactaagaactactccaactctgtgcaatggagagttccattaggtttgtgttttgcctgggctttgtttatgatcggtggtatgactttcgttccagaatccccacgttatttggttgaagctggtcaaattgacgaagcaagagcatctctttccaaagttaacaaggttgccccagaccatccattcattcaacaagagttggaagttattgaagctagtgttgaagaagctagagctgctggttcagcatcatggggtgagttgttcactggtaagccggccatgtttaagcgtactatgatgggtatcatgatccaatctctacaacaattgactggtgataactatttcttctactatggtactaccgtttttaacgctgttggtatgagtgattctttcgaaacttctattgttttcggtgtcgtcaacttcttctctacttgttgttctttgtacactgtcgatcgttttggacgtcgtaactgtttgttatatggtgccattggtatggtctgctgttatgtagtttacgcttctgttggtgtcaccagactatggccaaatggtgaaggtaatggttcatccaagggtgctggtaactgtatgattgtctttgcctgtttctatattttctgttttgctaccacttgggctccaattgcttatgttgttatttctgaaactttcccattgagagtcaagtctaaggctatgtctattgctacagctgctaattggttgtggggtttcttgattggtttcttcactccatttattactggtgctattaacttctactacggttacgttttcatgggctgtatggttttcgcctacttctacgttttcttctttgtgccagaaactaagggtttgactttggaagaagtcaatgatatgtacgctgaaggtgttctaccatggaagtctgcttcatgggttccaacatctcaaagaggtgctaactacgatgctgatgcattgatgcatgatgaccagccattctacaagaaaatgttcggcaagaaataa

SEQ ID NO: 26 - protein, 567 aa, Saccharomyces cerevisiae
MNSTPDLISPQKSSENSNADLPSNSSQVMNMPEEKGVQDDFQAEADQVLTNPNTGKGAYVTVSICCVMVAFGGFVFGWDTGTISGFVAQTDFLRRFGMKHKDGSYYLSKVRTGLIVSIFNIGCAIGGIILAKLGDMYGRKMGLIVVVVIYIIGIIIQIASINKWYQYFIGRIISGLGVGGIAVLSPMLISEVAPKEMRGTLVSCYQLMITLGIFLGYCTNFGTKNYSNSVQWRVPLGLCFAWALFMIGGMTFVPESPRYLVEAGQIDEARASLSKVNKVAPDHPFIQQELEVIEASVEEARAAGSASWGELFTGKPAMFKRTMMGIMIQSLQQLTGDNYFFYYGTTVFNAVGMSDSFETSIVFGVVNFFSTCCSLYTVDRFGRRNCLLYGAIGMVCCYVVYASVGVTRLWPNGEGNGSSKGAGNCMIVFACFYIFCFATTWAPIAYVVISETFPLRVKSKAMSIATAANWLWGFLIGFFTPFITGAINFYYGYVFMGCMVFAYFYVFFFVPETKGLTLEEVNDMYAEGVLPWKSASWVPTSQRGANYDADALMHDDQPFYKKMFGKK

SEQ ID NO: 27 - protein, 567 aa, Artificial Sequence
  mutated HXT3 protein
MNSTPDLISPQKSSENSNADLPSNSSQVMNMPEEKGVQDDFQAEADQVLTNPNTGKGAYVTVSICCVMVAFGGFVFGWDTGTISGFVAQTDFLRRFGMKHKDGSYYLSKVRTGLIVSIFNIGCAIGGIILAKLGDMYGRKMGLIVVVVIYIIGIIIQIASINKWYQYFIGRIISGLGVGGIAVLSPMLISEVAPKEMRGTLVSCYQLMVTLGIFLGYCTNFGTKNYSNSVQWRVPLGLCFAWALFMIGGMTFVPESPRYLVEAGQIDEARASLSKVNKVAPDHPFIQQELEVIEASVEEARAAGSASWGELFTGKPAMFKRTMMGIMIQSLQQLTGDNYFFYYGTTVFNAVGMSDSFETSIVFGVVNFFSTCCSLYTVDRFGRRNCLLYGAIGMVCCYVVYASVGVTRLWPNGEGNGSSKGAGNCMIVFACFYIFCFATTWAPIAYVVISETFPLRVKSKAMSIATAANWLWGFLIGFFTPFITGAINFYYGYVFMGCMVFAYFYVFFFVPETKGLTLEEVNDMYAEGVLPWKSASWVPTSQRGANYDADALMHDDQPFYKKMFGKK

SEQ ID NO: 28 - DNA, 1704 bp, Artificial Sequence
  mutated HXT3 gene
atgaattcaactccagatttaatatctccacaaaagtcaagtgagaattcgaatgctgacctgccttcgaatagctctcaggtaatgaacatgcctgaagaaaaaggtgttcaagatgatttccaagctgaggccgaccaagtacttaccaacccaaatacaggtaaaggtgcatatgtcactgtgtctatctgttgtgttatggttgccttcggtggtttcgttttcggttgggatactggtaccatttctggtttcgtcgcccaaactgatttcttgagaagattcggtatgaagcataaagatggtagttattatttgtctaaggttagaactggtttaattgtctccattttcaacattggttgtgccattggtggtattattttggctaaattgggtgatatgtacggtcgtaaaatgggtttgattgtcgttgttgttatctacatcatcggtattattattcaaattgcatccatcaacaaatggtaccaatatttcatcggtagaattatttccggtttgggtgttggtggtattgccgttttatctcctatgttgatttctgaagtcgctcctaaggaaatgagaggtactttagtctcctgttaccaactgatggttaccttgggtattttcttgggttactgtaccaacttcggtactaagaactactccaactctgtgcaatggagagttccattaggtttgtgttttgcctgggctttgtttatgatcggtggtatgactttcgttccagaatccccacgttatttggttgaagctggtcaaattgacgaagcaagagcatctctttccaaagttaacaaggttgccccagaccatccattcattcaacaagagttggaagttattgaagctagtgttgaagaagctagagctgctggttcagcatcatggggtgagttgttcactggtaagccggccatgtttaagcgtactatgatgggtatcatgatccaatctctacaacaattgactggtgataactatttcttctactatggtactaccgtttttaacgctgttggtatgagtgattctttcgaaacttctattgttttcggtgtcgtcaacttcttctctacttgttgttctttgtacactgtcgatcgttttggacgtcgtaactgtttgttatatggtgccattggtatggtctgctgttatgtagtttacgcttctgttggtgtcaccagactatggccaaatggtgaaggtaatggttcatccaagggtgctggtaactgtatgattgtctttgcctgtttctatattttctgttttgctaccacttgggctccaattgcttatgttgttatttctgaaactttcccattgagagtcaagtctaaggctatgtctattgctacagctgctaattggttgtggggtttcttgattggtttcttcactccatttattactggtgctattaacttctactacggttacgttttcatgggctgtatggttttcgcctacttctacgttttcttctttgtgccagaaactaagggtttgactttggaagaagtcaatgatatgtacgctgaaggtgttctaccatggaagtctgcttcatgggttccaacatctcaaagaggtgctaactacgatgctgatgcattgatgcatgatgaccagccattctacaagaaaatgttcggcaagaaataa

SEQ ID NO: 29 - DNA, 1704 bp, Artificial Sequence
  mutated HXT3 gene II
atgaattcaactccagatttaatatctccacaaaagtcaagtgagaattcgaatgctgacctgccttcgaatagctctcaggtaatgaacatgcctgaagaaaaaggtgttcaagatgatttccaagctgaggccgaccaagtacttaccaacccaaatacaggtaaaggtgcatatgtcactgtgtctatctgttgtgttatggttgccttcggtggtttcgttttcggttgggatactggtaccatttctggtttcgtcgcccaaactgatttcttgagaagattcggtatgaagcataaagatggtagttattatttgtctaaggttagaactggtttaattgtctccattttcaacattggttgtgccattggtggtattattttggctaaattgggtgatatgtacggtcgtaaaatgggtttgattgtcgttgttgttatctacatcatcggtattattattcaaattgcatccatcaacaaatggtaccaatacttcatcggtagaattatttccggtttgggtgttggtggtattgccgttttatctcctatgttgatttctgaagtcgctcctaaggaaatgagaggtgctttagtctcctgttaccaactgatggttaccttgggtattttcttgggttactgtaccaacttcggtactaagaactactccaactctgtgcaatggagagttccattaggtttgtgttttgcctgggctttgtttatgatcggtggtatgactttcgttccagaatccccacgttatttggttgaagctggtcaaattgacgaagcaagagcatctctttccaaagttaacaaggttgccccagaccatccattcattcaacaagagttggaagttattgaagctagtgttgaagaagctagagctgctggttcagcatcatggggtgagttgttcactggtaagccggccatgtttaagcgtactatgataggtatcatgatccaatctctacaacaattgactggtgataactatttcttctactatggtactaccgtttttaacgctgttggtatgagtgattctttcgaaacttctattgttttcggtgtcgtcaacttcttctccacttgttgttctctgtacaccgttgaccgttttggccgtcgtaactgtttgatgtggggtgctgtcggtatggtctgctgttatgttgtctatgcttctgttggagtcactagattatggccaaatggtcaaaacaacggctcatccaagggtgctggtaactgtatgattgtctttgcctgtttctatattttctgtttcgctactacctgggccccaattgcttatgtcgttgtttctgaaactttcccattgagagtcaagtctaaggctatgtctattgctacagctgctaactggatctggggtttcttgattggtttcttcactccatttattactggtgctattaacttctactacggttacgttttcatgggctgtatggttttcgcctacttctacgttttcttctttgtgccagaaactaagggtttgactttggaagaagtcaatgatatgtacgctgaaggtgttctaccatggaagtctgcttcatgggttccaacatctcaaagaggtgctaactacgatgctgatgcattgatgcatgatgaccagccattctacaagaaaatgttcggcaagaaataa

SEQ ID NO: 30 - protein, 567 aa, Artificial Sequence
  mutated HXT3 protein II
MNSTPDLISPQKSSENSNADLPSNSSQVMNMPEEKGVQDDFQAEADQVLTNPNTGKGAYVTVSICCVMVAFGGFVFGWDTGTISGFVAQTDFLRRFGMKHKDGSYYLSKVRTGLIVSIFNIGCAIGGIILAKLGDMYGRKMGLIVVVVIYIIGIIIQIASINKWYQYFIGRIISGLGVGGIAVLSPMLISEVAPKEMRGALVSCYQLMVTLGIFLGYCTNFGTKNYSNSVQWRVPLGLCFAWALFMIGGMTFVPESPRYLVEAGQIDEARASLSKVNKVAPDHPFIQQELEVIEASVEEARAAGSASWGELFTGKPAMFKRTMIGIMIQSLQQLTGDNYFFYYGTTVFNAVGMSDSFETSIVFGVVNFFSTCCSLYTVDRFGRRNCLMWGAVGMVCCYVVYASVGVTRLWPNGQNNGSSKGAGNCMIVFACFYIFCFATTWAPIAYVVVSETFPLRVKSKAMSIATAANWIWGFLIGFFTPFITGAINFYYGYVFMGCMVFAYFYVFFFVPETKGLTLEEVNDMYAEGVLPWKSASWVPTSQRGANYDADALMHDDQPFYKKMFGKK